(19) United States
(12) Patent Application Publication
(10) Pub. No.: US 2010/0318002 A1
(43) Pub. Date: Dec. 16, 2010

(54) Acoustic-Feedback Power Control During Focused Ultrasound Delivery

(76) Inventors: Oleg Prus, Haifa (IL); Rita Schmidt, Givataim (IL); Eyal Zadicario, Tel Aviv-Yafo (IL); Shuki Vitek, Haifa (IL)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/813,016

(22) Filed: Jun. 10, 2010

Related U.S. Application Data
(60) Provisional application No. 61/185,822, filed on Jun. 10, 2009.

(51) Int. Cl. A61N 7/00

(52) U.S. Cl. 601/2

(57) ABSTRACT

Ultrasound energy is delivered to a patient in a controlled manner using a focused ultrasound system, thus maintaining the desired therapeutic effect without causing unwanted damage to surrounding tissue. An ultrasound transducer device includes multiple transducer elements, each of which is controlled by drive circuitry and a drive signal controller. An acoustic detector detects signals indicative of cavitation in tissue targeted by the transducer elements, and the drive signal controller manages the delivery of acoustic energy from the transducer elements based on the detected cavitation signals such that a therapeutic effect at the target tissue remains within an efficacy range.

### CROSS-REFERENCE TO RELATED APPLICATIONS

This application claims priority to and the benefits of U.S. provisional patent application Ser. No. 61/185,822, filed Jun. 10, 2009, the entire disclosure of which is incorporated be reference herein.

### TECHNICAL FIELD

The present invention relates generally to systems and methods for performing noninvasive procedures using acoustic energy, and, more particularly, to systems and methods for limiting damage to healthy tissue during therapeutic delivery of ultrasonic energy.

### BACKGROUND INFORMATION

Diseased tissue, such as a benign or malignant tumor or blood clot within a patient's skull or other body region, may be treated invasively by surgically removing the tissue, or non-invasively by ablating or otherwise causing tissue necrosis using focused energy delivered from an external source. Both approaches may effectively treat certain localized conditions within the brain, for example, but require delicate performance to avoid destroying or damaging healthy tissue. These treatments may not be appropriate for conditions in which diseased tissue is integrated into healthy tissue, unless destroying the healthy tissue is unlikely to affect neurological function significantly.

Thermal ablation, as may be accomplished using focused ultrasound, has particular appeal for treating internal tissue because it generally does not disturb intervening or surrounding healthy tissue. Focused ultrasound may also be attractive, in that acoustic energy generally penetrates well through soft tissues, and ultrasonic energy, in particular, may be focused within zones having a cross-section of only a few millimeters; this is due to the relatively short wavelengths (e.g., as small as 1.5 millimeters (mm) in cross-section at one MegaHertz (MHz) of ultrasonic energy. Thus, ultrasound may be focused at a small target in order to ablate the tissue without significantly damaging surrounding healthy tissue.

As one example, low-frequency therapeutic ultrasound offers considerable advantages in trans-cranial brain treatments where skull heating is a risk. At the same time, however, at low frequencies the absorption of the acoustic energy by the tissue to be treated is very low. As a result, the preferred method of achieving thermal ablation relies on cavitation—i.e., the process by which microscopic bubbles are formed and implode violently, producing shock waves that destroy the target tissue. Unfortunately, cavitation is highly sensitive to local tissue characteristics and is difficult to model and predict in in-vivo. Without the ability to predict cavitation thresholds, too much or too little energy may be applied, resulting in insufficient energy being delivered to the target tissue, uncontrolled effects of excess cavitation such as expansion of the affected area beyond the planned volume and/or a shift (generally towards the transducer) of the treatment volume.

Accordingly, there is a need for automated systems and methods for effectively monitoring and controlling in real time the effects of cavitation occurring in tissue being treated using focused ultrasound.

### SUMMARY OF THE INVENTION

The present invention provides procedures and systems that facilitate non-invasive, focused ultrasound treatment using cavitation. In general, the technique uses a closed-loop approach such that immediate feedback regarding the extent of cavitation is provided to an operator or to an automatic control system. The objective is to direct ultrasound energy at the target tissue so as to cause cavitation within the tissue cells while avoiding the unwanted results of cavitation in surrounding tissue. A closed-loop control mechanism in accordance with the present invention may utilize acoustic detectors to monitor and/or record, in real-time, the acoustic activity occurring at the tissue being treated. Because cavitation emits a distinct acoustic signal, it can be detected before it becomes disruptive. Further, the signal may be analyzed to determine whether to increase or decrease the acoustic power of the transducers, or to influence other cavitation parameters. A real-time control loop ensures that sufficient acoustic power is delivered to the tissue to cause cavitation (and, thereby, destruction of target tissue) while keeping cavitation within safety limits so that uncontrolled effects do not occur.

In a first aspect, a focused ultrasound system includes an ultrasound transducer device having multiple transducer elements and drive circuitry coupled to the transducer elements. The system also includes an acoustic detector configured to detect signals indicative of cavitation in tissue being targeted by the transducer elements, and a drive signal controller coupled to the drive circuitry. The controller manages the delivery of acoustic energy based on the cavitation signals detected by the acoustic detector such that the therapeutic effect at the targeted tissue remains within an efficacy range, which, in some cases, may change over time as the ultrasound energy is delivered to the target tissue. The efficacy range is defined by an efficacy threshold and a safety ceiling.

In some embodiments, the acoustic detector includes one or more hydrophones for detecting the cavitation signals. In some cases, the detector process the cavitation signals and produces a cavitation signature, which may include various control parameters that are correlated with the therapeutic effect. The drive signal controller may modify the sonication pattern (e.g., increase or decrease the sonication power) of the ultrasound transducer if the control parameters indicate that the therapeutic effect is outside the efficacy range. In some cases, control parameters include a broadband median that represents the median amplitude of the cavitation signals over a sensed frequency band. In certain embodiments, the transducers operate at about 220 kHz and the cavitation signals fall within the frequency band spanning 50 kHz to 120 kHz.

In another aspect, a method for controlling ultrasound energy being delivered to a patient using a focused ultrasound system includes delivering focused ultrasound energy to a target tissue within the patient and detecting signals (e.g., acoustic signals) indicative of cavitation in the target tissue. Further, the acoustic energy delivered from transducer elements within the ultrasound system is managed and controlled in response to the detected cavitation signals such that a therapeutic effect remains within an efficacy range defined by a efficacy threshold and a safety ceiling.

In some embodiments, the cavitation signals are detected periodically during delivery of the ultrasound treatment. A cavitation signature including various control parameters correlated with the therapeutic effect may be produced from the cavitation signals, which in turn may be compared to the efficacy range. One such control parameter may include a broadband median as described above. The power provided to the ultrasound transducer may be increased if the control parameters indicated that the therapeutic effect is below the efficacy threshold, or, in other cases, may be decreased if the control parameters are observed to be above the safety ceiling. In certain embodiments, the transducers operate at about 220 kHz and the cavitation signals fall within the frequency band spanning 50 kHz to 120 kHz. The target tissue may be a lesion, tumor or other mass, and in some cases may be within the brain of the patient.

The foregoing and other objects, features and advantages of the present invention disclosed herein, as well as the invention itself, will be more fully understood from the following description of preferred embodiments and claims.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 schematically illustrates a system for monitoring physiological effects of ultrasound treatment in accordance with various embodiments of the invention.

FIG. 2 is a flow chart illustrating a method for administering ultrasound therapy in accordance with various embodiments of the invention.

FIG. 3 is a graphical representation of a signal detected during the administration of ultrasound therapy in accordance with various embodiments of the invention.

FIG. 4a is a graphical representation of a signal detected during the administration of ultrasound therapy as compared to various safety and efficacy thresholds.

FIG. 4b is a graphical representation of the effect of ultrasound therapy at a particular energy level over time.

### DETAILED DESCRIPTION

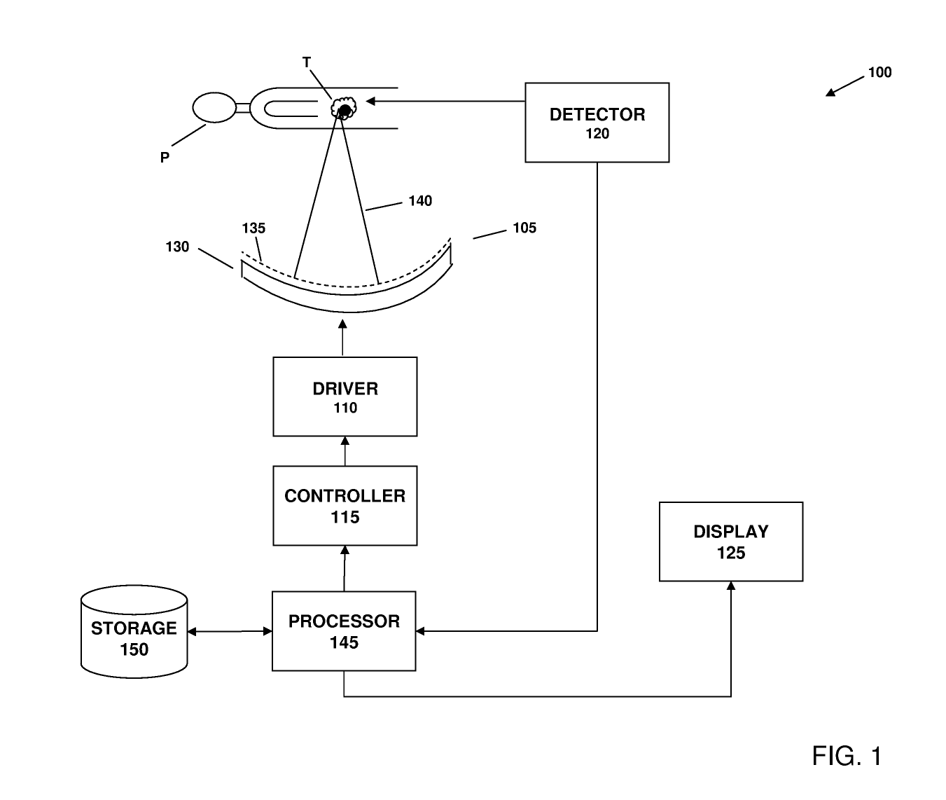

FIG. 1 illustrates one embodiment a system 100 for using focused ultrasound to treat tissue T within or upon a patient P. The system 100 includes a high-intensity focused-ultrasound phased-array transducer device 105, drive circuitry 110, a controller 115, and means for detecting signals emanating from the treated tissue 120. By monitoring (using, for example, a monitor or other display device 125) and processing the detected signals as part of a control feedback loop, the therapeutic effect of the focused ultrasound remains within an efficacy range.

The transducer device 105 is configured to deliver acoustic energy to target tissue T within or on a patient P. The acoustic energy may be used to coagulate, generate mechanical damage in, necrose, heat, cavitate or otherwise treat the target tissue T, which may be a benign or malignant tumor within an organ or other tissue structure.

In various embodiments, the transducer device 105 includes a mounting structure 130 and a plurality of transducer elements 135 secured to the structure 130. The structure 130 may have a curved shape in order to conform to various anatomical features of the patient, such as a skull. In other embodiments, the structure may have other shapes, forms, and/or configurations so long as it provides a platform or area to which the transducer elements 135 can be secured. The structure 130 may be substantially rigid, semi-rigid, or substantially flexible, and can be made from a variety of materials, such as plastics, polymers, metals, and alloys. The structure 130 can be manufactured as a single unit, or alternatively, be assembled from a plurality of components that are parts of the transducer device 105.

The transducer elements 135 are coupled to the drive circuitry 110 and a drive signal controller 115 for generating and/or controlling the acoustic energy emitted by the transducer elements 135. The transducer elements 135 may be coupled to the drive circuitry in a one-to-one manner (i.e., one circuit for each element) or in a many-to-one manner, in which multiple elements are controlled by a single circuit. Examples of such mappings are described in co-pending U.S. patent application Ser. No. 11/562,749, entitled “Hierarchical Switching in Ultra-High Density Ultrasound Arrays” the entire disclosure of which is incorporated herein by reference.

The transducer elements 135 convert the drive signals into acoustic energy, which may be focused using conventional methods. The controller drive circuitry 115 may be separate or integral components. It will be appreciated by those skilled in the art that the operations performed by the controller and/or drive circuitry may be performed by one or more controllers, processors, and/or other electronic components, including software and/or hardware components.

The drive circuitry, which may be an electrical oscillator, generates drive signals in the ultrasound frequency spectrum, e.g., as low as 50 kHz or as high as 10 MHz. Preferably, the driver provides drive signals to the transducer elements at radio frequencies (RF), for example, between about 100 kHz to 10 MHz (and more preferably between 200 kHz and 3.0 MHz), which corresponds to wavelengths of approximately 7.5 mm to 0.5 mm in tissue. However, in other embodiments, the driver can be configured to operate in other frequency ranges. When the drive signals are provided to the transducer elements 135, the elements emit acoustic energy from their respective emission surfaces, as is well known to those skilled in the art.

The controller 115 controls the amplitude, and therefore the intensity or power, of the acoustic waves transmitted by the transducer elements 135. In some embodiments, the controller 115 may also control a phase component of the drive signals to respective elements of the transducer device to control the shape or size of the focal zone 140 generated by the transducer elements and/or to move the focal zone to a desired location. For example, the controller may control the phase shift of the drive signals to adjust the distance from the face of the transducer element to the center of the focal zone (i.e., the “focal distance”). Specific examples of such an arrangement are described in U.S. Pat. No. 7,611,462, entitled “Acoustic Beam Forming in Phased Arrays Including Large Numbers of Transducer Elements” the entire disclosure of which is incorporated herein by reference.

In addition to the transducer elements and control circuitry, one or more acoustic detectors 120 may be integrated into or used with the focused ultrasound treatment apparatus to detect signals emanating from the target. In various embodiments, the detected signals include acoustic signals generated as a result of cavitation within the treated tissue T. Generally, cavitation is a phenomenon in which bubbles form within a liquid whose pressure falls below its vapor pressure. Cavitation describes two classes of behavior: inertial (or transient) cavitation, and non-inertial cavitation. Inertial cavitation refers to the rapid collapse of a void or bubble in a liquid, thus producing a shock wave. The acoustic signature of stable and inertial cavitation can be distinguished based on an analysis of the resulting acoustic signal. The acoustic signals produced by inertial cavitation can be sensed using one or more detectors such as hydrophones or other microphones designed to record or listen to sounds travelling through liquid or semi-solid mass. The detectors may be attached to the transducer assembly, or, in some cases, can be separate from the transducers. The signal (or signals) detected by the hydrophones may serve as input into a real-time control process algorithm executed on a processor 145 to determine whether the power supplied to the transducers should be increased or decreased. In some embodiments, the process algorithm uses a Fourier transform to transform the frequency-domain representation of the signal into a time-domain signal, which may then be compared to the efficacy range. Such a transformation is particularly beneficial in implementations where the efficacy range changes over time as the sonication is delivered to the patient and to identify the signature of the cavitation. In practice, the frequency domain signal may contain components of both inertial and stable cavitation simultaneously. The system may also include one or more storage devices 150 to store representations of the acoustic signals, threshold values, and/or results of the signal analysis algorithm.

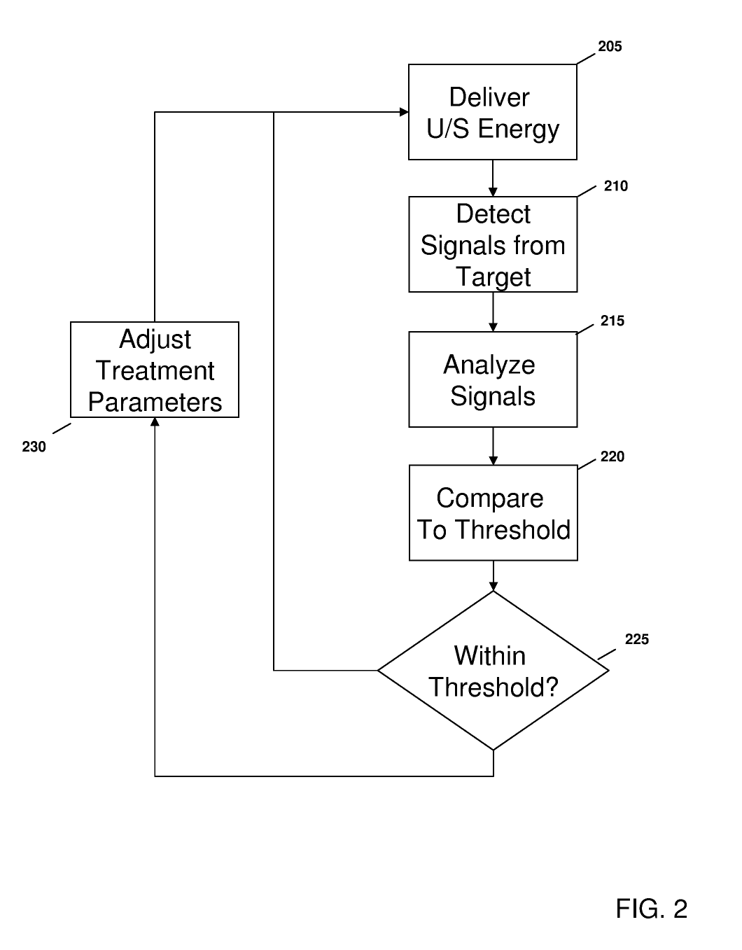

FIG. 2 illustrates one method implemented using the system described above. A patient is positioned on a table or other supporting device and an operator initiates treatment using a focused ultrasound system (STEP 205). The treatment may be delivered in a single sonication, multiple sonications during a single session, or during multiple sessions over time. In each case, the effect of the ultrasound on the cells within a target region are monitored using a detection device (STEP 210). The detection device may be, for example, an acoustic detection device such as a hydrophone that monitors sound waves released from the target tissue as cavitation occurs. Because different cavitation events have distinct acoustic properties, the monitored signals provide valuable information regarding the effect of the ultrasound energy at the target.

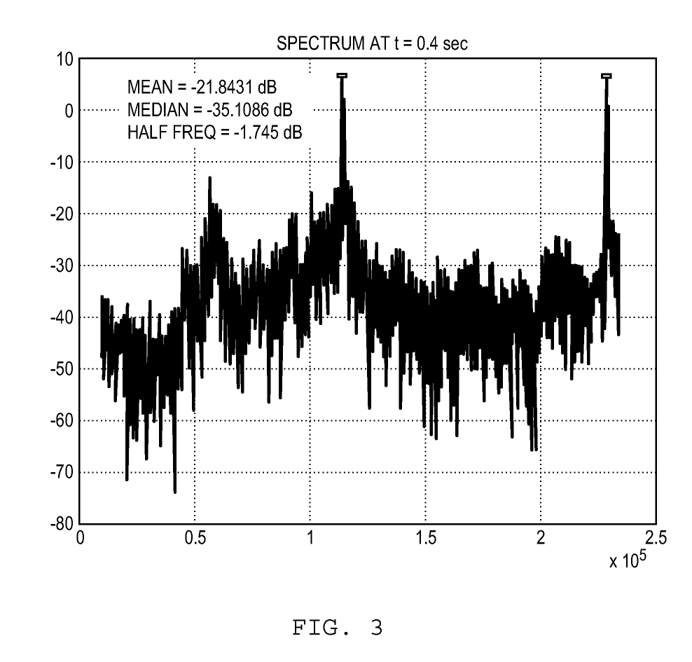

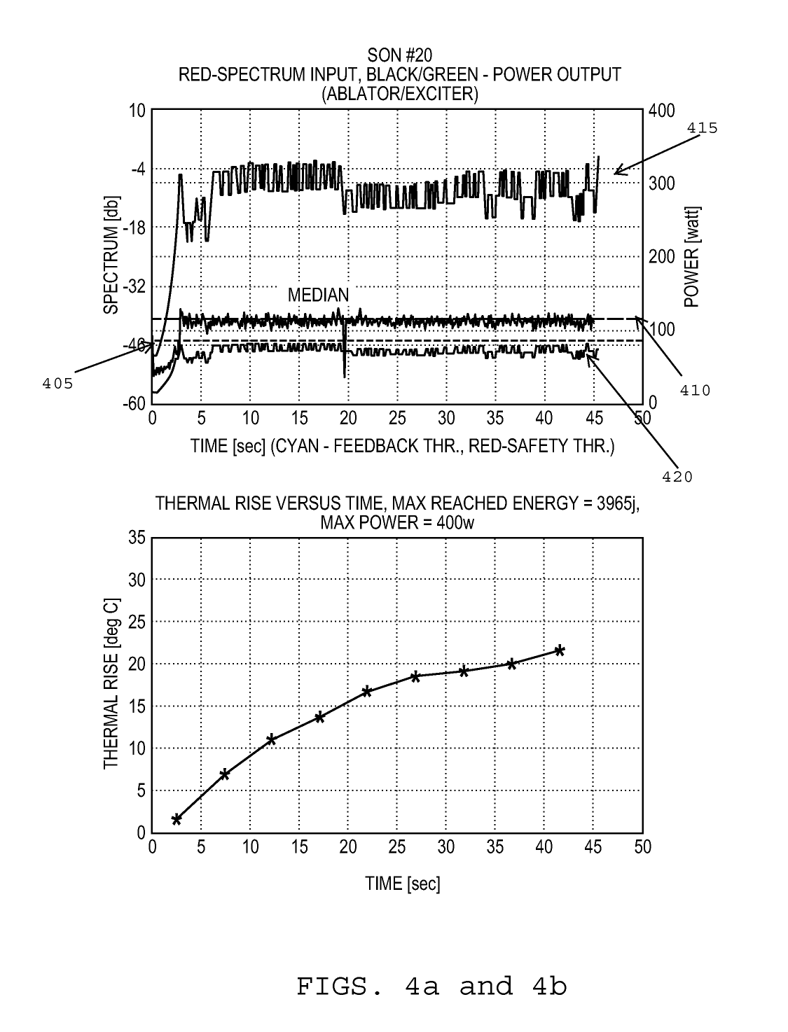

The acoustic signals are then analyzed (STEP 215) as described below with reference to FIGS. 3 and 4. For example, the acoustic signals may be compared (STEP 220) to an efficacy threshold to determine if the ultrasound energy being absorbed at the target is sufficient to cause the desired effects. The signals may also be compared to a safety threshold to ensure the amount of energy being delivered does not exceed a maximum. The comparisons may occur periodically during treatment, or, in some cases, at the end of a sonication. In either case, a determination is made (STEP 225) as to whether the signals are within the acceptable thresholds. If so, treatment continues uninterrupted. If, however, a threshold is violated, one or more treatment parameters may be adjusted (STEP 230). In some instances treatment may be halted in order to implement the changes, whereas in other cases the adjustments may be made in real-time as treatment continues.

FIG. 3 illustrates an exemplary signal indicative of cavitation occurring in tissue as detected over an acoustic frequency band as ultrasound energy is delivered to an intra-cranial tissue mass. The acoustic signal is analyzed for one or more specific cavitation signatures, e.g., in the frequency domain. The signatures may then be compared to target values and/or a efficacy ranges to determine if the acoustic energy being delivered to the target tissue is sufficient to initiate and maintain cavitation or if an undesired amount of cavitation is occurring.

In some cases, the acoustic signal is acquired throughout delivery of the ultrasound treatment according to a prescribed periodicity (e.g., every 30 msec). At each acquisition, a spectral analysis is computed and compared to the efficacy range. In cases where the characteristics imply that the cavitation level is below the effectiveness level, the drive controller increases the acoustic power delivered through the transducers. If the cavitation level is within the efficacy range, the driving power remains the same for the next cycle. If the cavitation level is close to or above the safety ceiling, the driving power is decreased. Variations of other parameters such as duration, frequency, excitation pulse, and duty cycle may also be used to affect cavitation in the treated tissue.

Referring to FIGS. 4a and 4b, the median amplitude of the cavitation signal may be measured over a sensed acoustic frequency band (a “broadband median”) and used as the (or one of the) control parameters indicating the therapeutic effect of the acoustic energy being delivered to the target tissue. The median may be computed and updated at every time interval (or every n^{th} interval) and compared with the efficacy threshold and/or the safety ceiling. In particular embodiments in which the transducer operates at 220 kHz, the spectral signal between 50 kHz-120 kHz is observed. In other embodiments, analysis of the spectral density of certain sub-harmonic signals and/or the use of a moving average window may be used to identify discreet spectral areas that present high spectral energy levels.

FIG. 4a illustrates the observed median during a typical sonication as bounded by the efficacy threshold 405 and the safety ceiling 410, each indicated as a horizontal bar. In this particular embodiment, two levels of sonication power is illustrated: an excitor level 415 that initiates cavitation and an ablator power level 420 that sustains the controlled cavitation. FIG. 4b illustrates a resulting thermal rise at a particular power level over time.

While the invention has been particularly shown and described with reference to specific embodiments, it should be understood by those skilled in the area that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

What is claimed is:

1. A focused ultrasound system, comprising:
an ultrasound transducer device having a plurality of transducer elements;
an acoustic detector configured to detect signals indicative of cavitation in tissue targeted by the transducer elements;
drive circuitry coupled to the transducer elements; and
a drive signal controller coupled to the drive circuitry, the drive signal controller controlling delivery of acoustic energy from the transducer elements based at least in part on the detected cavitation signals so that a therapeutic effect at the target tissue remains within an efficacy range defined by an efficacy threshold and a safety ceiling.

2. The system of claim 1 wherein the acoustic detector comprises one or more hydrophones.

3. The system of claim 1 wherein the acoustic detector produces a cavitation signature.

4. The system of claim 3 wherein the cavitation signature comprises one or more control parameters correlated with the therapeutic effect.

5. The system of claim 4 wherein the acoustic detector assesses whether the therapeutic effect is within the efficacy range based on the at least one control parameter and the correlation.

6. The system of claim 4 wherein the efficacy range changes as the acoustic energy is delivered.

7. The system of claim 4 wherein the plurality of transducers operate at about 220 kHz and the control parameters comprise a measurement of an acoustic signal between about 50 kHz and about 120 KHz.

8. The system of claim 4 wherein the control parameters comprise a broadband median representing the median amplitude of the cavitation signal over a sensed acoustic frequency band.

9. The system of claim 5 wherein the drive signal controller increases sonication power of the ultrasound transducer if one or more of the control parameters indicate that the therapeutic effect is below the efficacy threshold.

10. The system of claim 5 wherein the drive signal controller decreases sonication power of the ultrasound transducer if one or more of the control parameters indicate that the therapeutic effect is above the safety ceiling.

11. A method for controlling ultrasound energy being delivered to a patient using a focused ultrasound system that comprises a transducer having a plurality of transducer elements, the method comprising:
delivering, via the transducer, ultrasound energy to a target tissue within the patient;
detecting signals indicative of cavitation in the target tissue;
controlling delivery of acoustic energy from the transducer elements based at least in part on the detected cavitation signals so that a therapeutic effect at the target tissue remains within an efficacy range defined by an efficacy threshold and a safety ceiling.

12. The method of claim 11 further comprising detecting the signals according to a prescribed periodicity.

13. The method of claim 11 wherein the signals are acoustic signals.

14. The method of claim 11 further comprising producing a cavitation signature based on the detected cavitation signals, the cavitation signature comprising one or more control parameters correlated with the therapeutic effect.

15. The method of claim 14 wherein the one or more control parameters comprises a broadband median representing the median amplitude of the cavitation signal over a sensed acoustic frequency band.

16. The method of claim 15 further comprising assessing whether the therapeutic effect is within the efficacy range based at least in part on the broadband median.

17. The method of claim 16 further comprising increasing power to the ultrasound transducer if one or more of the control parameters indicate that the therapeutic effect is below the efficacy threshold.

18. The method of claim 16 further comprising decreasing power to the ultrasound transducer if one or more of the control parameters are above the safety ceiling.

19. The method of claim 11 wherein the plurality of transducer elements operate at about 220 kHz and the control parameters comprise a measurement of a signal between about 50 kHz and about 120 KHz.

20. The method of claim 11 wherein the tissue to be treated comprises brain tissue.
